(19) United States
(12) Patent Application Publication
(10) Pub. No.: US 2011/0009836 A1
(43) Pub. Date: Jan. 13, 2011

(54) PHARMACEUTICAL DELIVERY DEVICE

(75) Inventors: Chafic Chebli, Pointe-claire (CA); Hanna Piskorz, Richmond Hill (CA)

Correspondence Address: STIKEMAN ELLIOTT LLP, 1600-50 O''CONNOR STREET, OTTAWA, ON, K1P 6L2, CA

(73) Assignee: PHARMASCIENCE INC., Montreal, QC (CA)

(21) Appl. No.: 12/809,498

(22) PCT Filed: Dec. 18, 2008
(86) PCT No.: PCT/CA08/02182

§ 371 Date: Jun. 18, 2010

(30) Foreign Application Priority Data
Dec. 21, 2007 (CA) 2616392

(51) Int. Cl. A61F 9/00

(52) U.S. Cl. 604/298

(57) ABSTRACT

The present application discloses a pharmaceutical delivery system for delivering a desired amount of liquid in the form of droplets. The pharmaceutical delivery system comprises a container having resilient compressible walls of uniform thickness, an elongated neck, and a valveless dropper tip. The pharmaceutical delivery system dispenses liquid through an outlet orifice in the valveless dropper tip in a drop by drop manner, producing droplets of substantially uniform volume independent of the amount of pressure applied on the container.

### FIELD OF THE INVENTION

The present invention relates to a pharmaceutical delivery device for drop dispensing liquid medicament. More particularly, the present invention relates to a liquid delivery device with the ability to dispense fluid in a drop by drop manner with uniform droplet volume.

### BACKGROUND OF THE INVENTION

Typical pharmaceutical delivery devices, such as dropper bottles for administering ophthalmic fluid, are well known in the prior art. Generally, these liquid delivery devices comprise a medicament reservoir, medicament delivery passage and a drive mechanism for metering or flow regulation of liquid adjusted to transfer a measured amount of dose. The drive mechanism can be gas, pressure activated, hot water activated, piston, valve, electric impulse, electrolyte, cartridge, ball and plunger, etc.

The present application is focused on liquid delivery devices that are “pressure activated”, otherwise known as squeeze bottles or dropper bottles. These devices generally consist of a squeezable container with a tapered dispenser that terminates in a discharge aperture (i.e. a dropper tip). For example, to administer ophthalmic fluid, the discharge aperture is aligned above a target eye and the bottle is squeezed to urge out a drop or dose of the fluid.

Although the conventional design is widely used, it suffers from several drawbacks. Primarily, dose volume is difficult to repeatedly control, in part, because a proper amount of squeeze force is difficult to repeatedly apply to the dropper bottle.

According to European Patent No. 0,416,694 issued Jul. 13, 1994 (Bunin), the conventional ophthalmic drug delivery system consists of a dropper tip and squeeze bottle, intended to deliver the medication in a drop by drop manner, the drops being of a uniform predetermined size. The drop size control is achieved by means of the hydraulic resistance presented by a small diameter channel forming the beginning of a straight delivery channel in the dropper tip, having a diameter of about 0.006 inches or 0.150 mm, which restricts the flow from the squeeze bottle. This conventional drug delivery system generally serves the intended purpose, but, as the Bunin reference notes, in the hands of many patients it does not always result in discrete drops but rather streaming of the liquid resulting in loss of medication and possibly soiled clothes and fabric. This is also known as the streaming effect.

U.S. Pat. No. 4,584,823 issued Apr. 29, 1986 (Nagel) discloses a method of blow molding in a single form-fill-seal operation a droplet dispenser bottle which delivers fluid in precise, uniform drops rather than streamlets. The dispenser formed by this method was intended to dispense the desired amount of fluid from the bottle regardless of how hard the bottle is squeezed. The dropper tip of the dispenser disclosed in Nagel has a first orifice, having a diameter of 0.004 inches, which is a straight delivery channel in communication with the container and a second orifice. The first orifice is proportioned to prevent a stream of fluid from being shot out of the bottle. The second orifice is cone shaped, and dispenses the drop.

The Bunin reference, discussed above, attempts to address the streaming effect problem by designing a dropper tip wherein the entry to the delivery channel is not through a narrow channel, but rather through an orifice, which is not concentric with the delivery channel but rather at an angle to it (preferably at a right angle to the delivery channel).

Another approach of achieving the desired result of dispensing a liquid medication in a drop by drop manner while avoiding streaming is to make use of a valve. For example, U.S. patent application Ser. No. 10/570,271, filed Aug. 30, 2004 (Kawashiro et al.), is directed to a delivery device that is capable of preventing a content liquid from flowing back into the delivery device, thus achieving aseptic delivery of a liquid drop. This delivery device makes use of, among other things, a valve to close the outlet orifice when there is no liquid pressure applied to the device.

Japanese Patent Application No. JP2000210368, filed Jan. 20, 1990 (Misaka), is directed to a constant pressure and constant delivery eye dropper. To achieve this result, the eye dropper is provided with a cap connecting part in an elastically deformable main body of the dropper in which a jet cylinder is inserted therein, a valve in the container of the jet cylinder and a liquid pool at the terminal part. Thus, the eye lotion is moved into the liquid pool by inclining the main body of the dropper, a bending part of the main body of the dropper is pressed and deformed, the inner pressure is increased and the eye lotion is discharged from the discharge hole by operating the valve so that the eye lotion can be easily dropped in the eyes with the constant pressure and constant delivery.

U.S. patent application Ser. No. 10/523,516, filed Aug. 6, 2003 (Cohen et al.), is directed to a dropper bottle capable of administering single doses or drops in a repeatable manner. This application addresses the shortcomings in the prior art by proposing various dropper bottle designs and dropper bottle assemblies including accessories for dropper bottles. For example, a cradle which is formed to be mounted onto a dropper bottle, wherein the free end of the cradle lever is pressed towards the bottle when it is in use, and a portion of the cradle indents a side of the bottle, thus controlling the amount of squeeze force applied. In addition, Cohen et al. discloses a delivery device having a webbed structure formed inside the dropper bottle which limits deformation of the bottle.

Another representation of a drug dispenser using a valve can be seen in FIGS. 1, 2, 3A and 3B, identified as “PRIOR ART”. The pharmaceutical delivery device disclosed is a blow fill seal (“BFS”) dispenser 1 comprising a container portion 2 (or reservoir) and neck portion 4, in which an overcap 6 can be screwed thereon. The dispenser is known to achieve the desired result of a dispensing a liquid in a drop by drop manner, and avoids any streaming effect, by making use of a valve 8. It can be seen from the aforementioned drawings that the valve 8 primarily consists of the base of the annular collar (containing a lip) 10 and a flexible nozzle tip 12, which enables a certain amount of liquid to escape from the reservoir when the walls of the dispenser are compressed.

As can be seen in FIG. 3A, when no pressure is applied to the dispenser (i.e. when it is not in use), the base of the flexible nozzle tip 14, which is located along the central axis of the dispenser, comes into contact with other portion (or lip) of the annular collar 10, thereby functioning as a valve and not allowing any liquid to escape from the dispenser. When the valve is in this position, it's in a closed position.

As can be seen in FIG. 3B, when pressure is applied to the walls of the dispenser, the liquid forces the flexible nozzle tip 8 to lift from the lower position of annular collar 10, and flex towards the other portion of the annular collar, thereby allowing a certain amount of liquid to be dispensed before the flexible nozzle tip 8 returns to its original position. When liquid escapes from the reservoir, the valve is in an open position. It is this particular mechanism that allows the illustrated prior art dispenser to dispense a liquid in drop by drop manner.

A common feature among these prior art pharmaceutical delivery devices is that they are designed to administer a constant volume of liquid. However, there are some drawback associated with prior art dispensers, for example, some consist of complex designs, require specialized dropper tips, are expensive to manufacture, are difficult to use and/or they have single function of use e.g. can be applied for delivery of liquids to eye, nose or skin only, etc.

There is therefore a need for a pharmaceutical delivery device that overcomes these aforementioned drawbacks and at the same time is simple in design, easy to use and also has a potential for multiple uses, for example, the same device could be filled with a liquid containing medicament to be administered to eye, ear, nose or skin.

The applicant of the present invention has developed a pharmaceutical delivery device that is simple in design, and is able to dispense fluid in a drop by drop manner while providing drops of substantially constant size, regardless of the amount of pressure placed on the container.

While the invention has been described in conjunction with illustrated embodiments thereof, it is evident that many alternatives, modifications and variations will be apparent to those skilled in the art of the foregoing description. Accordingly, it is intended to embrace all such alternatives, modifications and variations as full within the spirit and broad scope of the invention.

### SUMMARY OF THE INVENTION

According to an aspect of the present invention, there is a provided a pharmaceutical delivery system that solves many of the deficiencies present in the prior art delivery devices, making it possible to form a drop of substantially constant size for a given liquid even if the aforementioned system is tilted when the drop is formed, and regardless of the amount of pressure placed on the container.

In one embodiment of the present invention, there is provided a pharmaceutical delivery device for delivering a desired amount of liquid in the form of droplets of substantially constant size, said device comprising:
a container having a base, compressible walls of a predetermined thickness, and an elongated neck; and
a valveless dropper tip containing an upper and bottom portion, said upper portion having a narrow central channel extending towards an outlet orifice, and a lower portion in communication with the container via the elongated neck; whereby when the container is compressed the liquid contained therein is dispensed in a drop by drop manner through the outlet orifice of the dropper tip, independently on the amount of pressure applied onto the container.

In another aspect of the present invention, the predetermined thickness of the walls of the pharmaceutical delivery device are within the range of about 0.80 mm to about 1.35 mm thick.

Other advantages and features of the present invention will be more readily apparent from the following detailed description of the preferred embodiment of the invention, the accompanying drawings, and the appended claims.

### BRIEF DESCRIPTION OF THE DRAWINGS

The embodiments of the present invention are described below with reference to the accompanying drawings in which:

FIG. 1, identified as “PRIOR ART”, is a perspective view of a known dispenser;

FIG. 2, identified as “PRIOR ART”, is a sectional view of the dispenser of FIG. 1;

FIG. 3A, identified as “PRIOR ART” is a sectional view of the neck of the dispenser of FIG. 1 in a closed position;

FIG. 3B, identified as “PRIOR ART” is a sectional view of the neck of the dispenser of FIG. 1 in an open position;

FIG. 4 is a perspective view of an embodiment of the pharmaceutical delivery disclosed in the present application;

FIG. 5 is a sectional view of the pharmaceutical delivery device of FIG. 4;

FIG. 6 is a sectional view of the bottom of the pharmaceutical delivery device of FIG. 4; and

FIG. 7 is a sectional view of the bottom of the pharmaceutical delivery device of FIG. 4, when compressed.

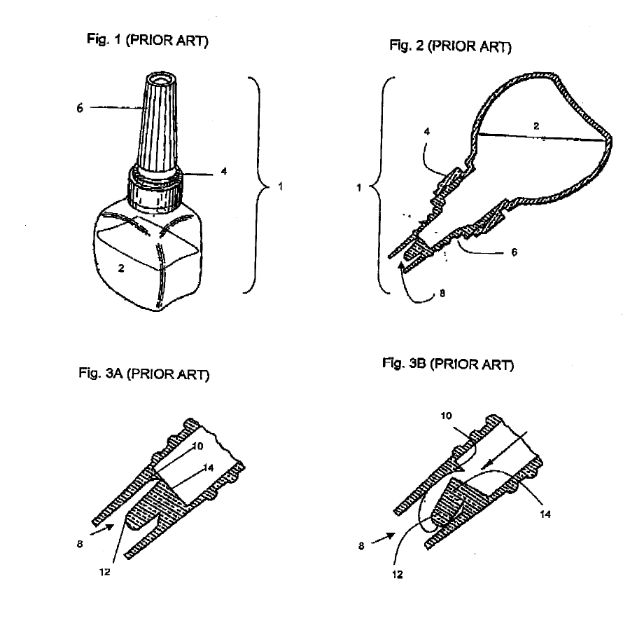

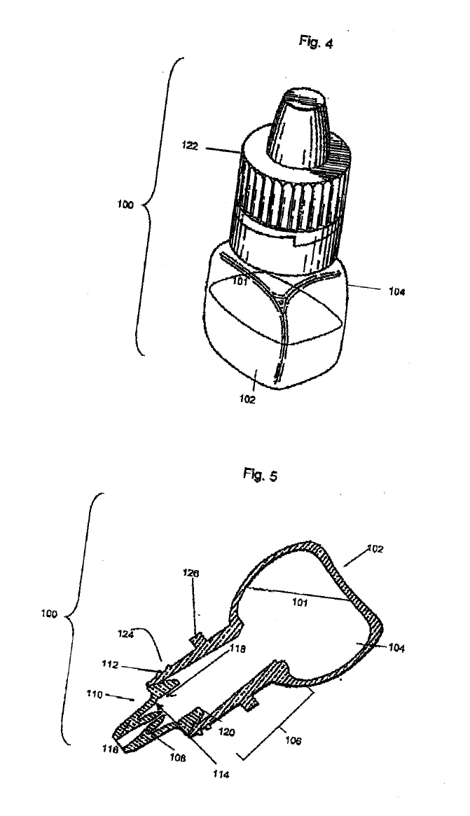

### DETAILED DESCRIPTION OF THE PREFERRED EMBODIMENTS

It has been surprisingly found that the pharmaceutical delivery device according to the present invention can dispense a liquid in a drop by drop manner and provide droplets of substantially constant volume, regardless the amount of pressure applied onto the container.

The combination of the shape of the pharmaceutical delivery device, in particular the container, and the thickness of the container's compressible walls, as well as the valveless dropper tip, including the narrow central channel and outlet orifice, allow the pharmaceutical delivery device of the present application to dispense single droplets in both a reproducible and uniform manner, independently of the pressure applied onto the compressible walls.

The preferred embodiment of the pharmaceutical delivery device 100 of the present application is illustrated in FIGS. 4 to 7. The pharmaceutical delivery device 100 is comprised of a container 101, having a base 102, which is preferably flat in relation to the surface on which the pharmaceutical delivery device sits. Formed to the base 102, and extending upward therefrom are compressible walls 104, which form the reservoir in the container 101. The compressible walls 104 are preferably convex, and the compressible walls are of a uniform, pre-determined thickness. The pharmaceutical delivery device 100 of illustrated embodiment the illustrated embodiment generally contains about 5 ml of medicament, and has a capacity ranging between about 5.5. to about 7.6 ml.

FIGS. 5 to 7 illustrate, as was noted above, that each of the compressible walls 104 are of uniform thickness. Indeed, in the manufacturing process it is important to control the thickness of the compressible walls 104. If the compressible walls are made too thin the undesired liquid streaming effect could occur, as the pressure exerted by the user on the container would not be efficiently dissipated over the surface of the container. Furthermore, the droplet size produced from such a container may not be of substantially uniform size as the amount of pressure required by the user to exert on the container to produce a droplet will be much lower, thus producing smaller droplets when little pressure is used, and larger droplets (or potentially streams of liquid, as noted above), when larger pressure is exerted on the container. On the other hand, if the compressible walls 104 are too thick, it will become too difficult to apply pressure on the container so as dispense a drop of liquid contained therein. Thus, it is imperative that the right compressible wall 104 thickness be achieved. The thickness of the compressible walls 104 is preferably in the range of about 0.50 mm to about 1.50 mm thick, more preferably, in the range of about 0.75 mm to 1.40 mm thick, and even more preferably, in the range of about 0.85 mm to about 1.30 mm thick. The use of these components in the manufacture of the container is well known to a person skilled in the art. However, the thickness of the compressible walls 104, as well as the overall shape of the pharmaceutical delivery device (i.e. the convex compressible walls 104), in combination with the valveless dropper tip 108, discussed in more detail below, function cooperatively to allow the pharmaceutical delivery device of the present application to deliver the liquid in a drop by drop manner, producing droplets of substantially uniform size, regardless of the pressure placed on the container. The width of the illustrated embodiment of the pharmaceutical deliver device 100, from the widest point of the convex compressible walls 104, is approximately 25.4 mm.

In the illustrated embodiment, as can be seen in FIG. 5, formed to each of the compressible walls, and extending upward therefrom, is an elongated neck 106. The elongated neck 106 is preferably in the shape of a cylinder, and in the illustrated embodiment, the overall internal diameter of the elongated neck 106 is in the range of about 8.0 to 10.0 mm. In the illustrated embodiment, the pharmaceutical delivery device 100 has a height, from the base 102 to the top most portion of the elongated neck 106, in the range of about 40.0 to about 48.0 mm, and the distance from the base 102 to the top of the compressible walls 104 (i.e. the point at which the elongated neck 106 extends from) is in the range of about 22.0 mm to about 24.0 mm.

Positioned in, and/or attached to, the elongated neck 106, is a valveless dropper tip 108. The valveless dropper tip 108 contains an upper portion 110 and bottom portion 112. The upper portion 110 comprises a narrow central channel 114, which is in flow communication with the container via the elongated neck 106. The narrow central channel 114 extends upwards towards an outlet orifice 116. At the lower extremity of the narrow central channel (i.e. the portion communicating with the elongated neck and subsequently with the container), is a small aperture 118. By way of its nature (or construction), the aperture 118 can only intake a certain volume of liquid corresponding, for example, to one drop or dosage, which will be expelled through the upper portion of the pharmaceutical delivery device of the present invention. When the compressible walls 104 of the container 101 are squeezed by the user (i.e. compressed) the liquid contained therein is dispensed in a drop by drop manner through the outlet orifice of the valveless dropper tip to provide droplets of substantially uniform volume, independent of the amount of pressure applied on the container.

As can be seen in FIG. 5, in the preferred embodiment, the outlet orifice 116 is in the shape of an inverted cone. In this connection, the converging portion of the outlet orifice communicates with the narrow central channel 114, and at the other extremity of said outlet orifice constitutes the diverging portion. Preferably, the diverging portion of the outlet orifice 116 has a diameter in the range of about 1.5 mm to about 3.0 mm.

In a preferred embodiment, the valveless dropper tip may be calibrated; thus capable of constantly delivering a predetermined amount of solution (i.e. droplet volume) in a drop by drop manner.

As it can be seen in FIG. 4, the bottom portion 112 of the valveless dropper tip 108 depends into the elongated neck 106. In the illustrated embodiment, the bottom portion 112 of the valveless dropper tip 108 can be slidingly engaged into the elongated neck 106. Alternatively, the valveless dropper tip 108, elongated neck 106, and container 101, may constitute a single piece (i.e. BFS). Nevertheless, if the valveless dropper tip 108 is to be slidingly engaged (or securably fixed) to the elongated neck 106 of the container 101, interlocking means 120 may be required. In this connection, many different types of interlocking means can be foreseen, such as corrugated protrusions, and would be readily apparent to the person of ordinary skill in the art.

As aforementioned, when the pharmaceutical delivery device is in use, a liquid travels from the container, through the aperture, into the narrow central channel and is eventually expelled (or dispensed) via the outlet orifice.

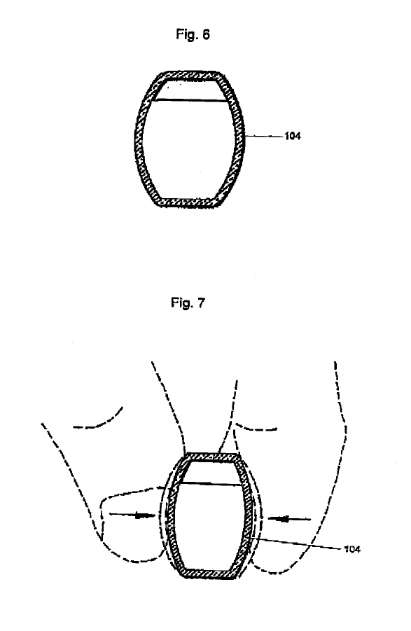

FIG. 6 and FIG. 7 illustrate the position of the compressible walls 104, when they are in a rest position (FIG. 6) and when they are compressed by the user (FIG. 7) to dispense a drop of the liquid contained within the container 101 of the pharmaceutical delivery device 100. By exerting pressure on the container 101, liquid contained therein is pushed upwards, through the elongated neck 106 and into the valveless dropper tip 108, which is in direct communication with the two latter components (i.e. the container and the elongated neck).

Referring once again to FIG. 4, the pharmaceutical delivery device 100 according to the present application may further comprise an overcap 122. In the illustrated embodiment, the overcap can be connected (or attached) to the outer portion of the elongated neck 106 of the container 101 by being screwed onto the top of the elongated neck 106, via threads 124 on the outer surface of the elongated neck 106 (in the illustrated embodiment, the outer thread diameter is in the range of about 13.5 mm to about 15.1 mm). Further, the elongated neck 106 can comprise a ledge 126 extending outwards from the outer surface of the elongated neck 106, and extending perpendicularly thereto, thus forming a collar or ring around the outer circumference of the elongated neck which acts as a ledge/stopper for the overcap 122 when it is fastened onto the pharmaceutical delivery device 100. In the illustrated embodiment, the distance from the top of the ledge 126 to the top of the elongated neck 106, is about 14.0 to 16.0 mm. The overcap 122 would protect the valveless dropper tip 108 from external contamination. Alternative means for attaching an overcap, such as snap-on, would be apparent to the person of ordinary skill in the art.

The pharmaceutical delivery device according to the present application 100 could also be provided with tamper evident features, such as security tape or cellophane, to ensure that pharmaceutical delivery device has not been previously opened or used. Such features are well known to a person skilled in the art, and could include a peel-off ring or a security seal, which can be fitted around the elongated neck 106 of the container.

The pharmaceutical delivery device 100 of the present application, including the container and the valveless dropper tip, is preferably made of a flexible plastic material, for example, low density polyethylene (LDPE), polyethylene (PE), polypropylene (PP) and the like, and can be prepared by any suitable technique, such as blow molding. It is to be understood that the present invention is not limited to the specific material from which the delivery device of the present invention is made, or the particular process by which it is made, as it will be understood by those skilled in the art that many different materials and various manufacturing techniques may be employed.

The choice of materials to be used in the manufacture of the pharmaceutical delivery device 100 is also dependent on the contents (i.e. liquids, etc.) the device is intended to be used with, as the contents may react with the materials used in the manufacture of the pharmaceutical delivery device 100. For example, it is known that certain ophthalmic solutions containing active medicaments, such as Latanoprost, adhere to most plastics. As such, it is important that a resin be used in the manufacture of the pharmaceutical delivery device 100 in order to avoid the active medicament adhering to the delivery device. Lupolen 1810E is a suitable resin for the bottle to remedy this problem. In a similar fashion, the Applicant used the resin Lupolen 1840H for a valveless dropper tip. These resins were manufactured by Basell in Europe. While Lupolen, as choice of resin, is preferably used, other resins can be used, and such resins are known to a person skilled in the art.

The liquid contained within the container of the pharmaceutical delivery device according to the present application may include any type of liquid known to the person skilled in the art, including physiologically acceptable ophthalmic liquid. The liquid may also contain at least one pharmaceutically active substance. Pharmaceutically active substances may include, though are not limited to, those described in the Compendium of Pharmaceuticals and Specialties (CPS), the International nonproprietary names (INN) lists of the World Health Organization (WHO), and in the International, European and/or U.S. Pharmacopoeias. In a preferred embodiment, the liquid contains the active substance, Latanoprost.

Furthermore, the pharmaceutical delivery device of the present application can be used for administration of drugs for localised or non-invasive mucosal systemic delivery of drugs. It is an endeavour of the present invention to encompass all these features.

Another aspect of the present invention is that the device can be used for immediate or rate controlled delivery of a variety of liquid products for non-invasive mucosal drug delivery application such as nose, ear, eye, skin and also have a particularly compact size.

The rheological behaviour of the product filled into the device can be selected based on the therapeutic requirement of the product. However, it is particularly preferred to deliver the liquid to the eye.

The drugs may be a solid, such as a tablet or powder, in solution and/or suspension in a pharmaceutically acceptable medium which is in liquid state or the drugs may themselves be liquids.

The drugs may be polar or non-polar medicaments of appropriate sizes suitable to treat illness conditions such as diabetes, cardio vascular/central nervous system disorders or imperfections, pain management, antibiotic therapy etc. but not limited to the same.

If desired, the walls of the container can be transparent or translucent, so that the amount of liquid product remaining can easily be ascertained. The liquid product may be coloured to make this determination easier.

### EXAMPLES

In bottle development, the Applicant analyzed a plurality of bottles, each bottle varying in compressible wall thickness, to obtain a substantially consistent droplet size. As it can be seen from TABLE 1, each of the bottles examined produced droplets of substantially uniform droplet size, with the 3.5 g bottle, which has compressible walls in the range of about 0.85 to about 1.30 mm in thickness, had the least variation in droplet size, and thus, more readily produces substantially consistent droplet volumes in a drop by drop manner. It is thus apparent that by selecting the right compressible wall thickness, and consequently the right bottle density, the more reproducible becomes the droplet volume.

TABLE 1

| | Bottle 1 | Bottle 2 | Bottle 3 | Bottle 4 | Bottle 5 | Variation |
|---|---|---|---|---|---|---|
| 3.5 | 0.0283 | 0.0282 | 0.0279 | 0.0281 | 0.0289 | 0.0010 |
| Gram | ml/ | ml/ | ml/ | ml/ | ml/ | ml/ |
| Bottle | drop- | droplet | droplet | droplet | droplet | droplet |
| | let | | | | | |
| 3.2 | 0.0264 | 0.0275 | 0.0279 | 0.0273 | 0.0291 | 0.0027 |
| Gram | ml/ | ml/ | ml/ | ml/ | ml/ | ml/ |
| Bottle | drop- | droplet | droplet | droplet | droplet | droplet |
| | let | | | | | |
| 2.9 | 0.0287 | 0.0287 | 0.0287 | 0.0274 | 0.0296 | 0.0022 |
| Gram | ml/ | ml/ | ml/ | ml/ | ml/ | ml/ |
| Bottle | drop- | droplet | droplet | droplet | droplet | droplet |
| | let | | | | | |

The Applicant also tested different dropper tips in order to obtain the droplet sizes in the desired range, when the bottle comprising compressible walls of appropriate thickness was used.

From the above, it may be appreciated that the present invention provides the patient and physician with an improved device for dispensing liquids

In a preferred embodiment, to use the pharmaceutical delivery system, a user usually breaks off the security seal of the pharmaceutical delivery device, and then unscrews the overcap 122. After such has been done, the user places the pharmaceutical delivery device 100 over his or her eye and applies pressure onto the walls 104 of the container (see FIG. 7) until a single drop of liquid is expelled from the device. The user may apply as many drops as required or as prescribed by the physician, for a given treatment.

The present invention is illustrated herein by example, and various modifications may be made by a person of ordinary skill in the art. For example, although the preferred operation of the device has been described above in connection with dispensing fluid medicine to the eye, the device may be used to dispense fluid medicine topically to other portions of the body, such as skin, ears or nose.

It is believed that the operation and construction of the present invention will be apparent from the foregoing description. While the device and methods shown or described above have been characterized as being preferred, various changes and modifications may be made therein without departing from the spirit and scope of the invention as defined in the appended claims.

1. A pharmaceutical delivery device for delivering droplets of liquid in substantially uniform volume, said device comprising:
a container having
a base;
compressible and resilient walls extending upwards from the base;
each of the walls having a uniform, predetermined thickness;
an elongated neck formed to each of the walls and extending upward therefrom;
a valveless dropper tip positioned in and/or attached to the elongated neck;
the valveless dropper tip having an upper and lower portion;
the upper portion having a narrow central channel extending towards an outlet orifice, and a lower portion in communication with the container via the elongated neck; whereby when the container is compressed the liquid contained therein is dispensed in a drop by drop manner through the outlet orifice of the dropper tip, producing droplets of substantially uniform volume independent of the amount of pressure applied to the container.

2. The pharmaceutical delivery device according to claim 1, wherein the predetermined thickness of the compressible and resilient walls is in the range of about 0.50 mm to about 1.50 mm thick.

3. The pharmaceutical delivery device according to claim 1, wherein the predetermined thickness of the compressible and resilient walls is in the range of about 0.75 mm to about 1.40 mm thick.

4. The pharmaceutical delivery device according to claim 1, wherein the predetermined thickness of the compressible and resilient walls is in the range of about 0.85 mm to about 1.30 mm.

5. The pharmaceutical delivery device according to claim 1, wherein the outlet orifice of the valveless dropper tip has a diameter in the range of about 1.5 mm to about 3.0 mm.

6. The pharmaceutical delivery device according to claim 1, wherein the elongated neck has means for receiving overcaps, thus protecting the dropper tip from external contamination.

7. The pharmaceutical delivery device according to claim 6, further comprising an overcap connectable onto the elongated neck of the container.

8. The pharmaceutical delivery device according to claim 7, wherein the overcap contains tamper evident features.

9. The pharmaceutical delivery device according to claim 1, wherein the container is made of polyethylene, the valveless dropper tip is made of polyethylene, and the overcap is made of polypropylene or any other suitable polymer material.

10. The pharmaceutical delivery device according to claim 1, wherein the pharmaceutical delivery device is manufactured in a translucent or transparent material.

11. The pharmaceutical delivery device according to claim 1, wherein the pharmaceutical delivery device may be used to administer medications as systemic non-invasive mucosal delivery system.

12. The pharmaceutical delivery device according to claim 1, wherein the pharmaceutical delivery device may be used to administer medications as localized mucosal delivery system.

13. The pharmaceutical delivery device according to claim 1, wherein the pharmaceutical delivery device may be used to administer medications for immediate or rate controlled delivery of medicaments.

14. The pharmaceutical delivery device according to claim 4, wherein the outlet orifice of the valveless dropper tip has a diameter in the range of about 1.5 mm to about 3.0 mm.

15. The pharmaceutical delivery device according to claim 5, wherein the elongated neck has means for receiving overcaps, thus protecting the dropper tip from external contamination.

16. The pharmaceutical delivery device according to claim 15, further comprising an overcap connectable onto the elongated neck of the container.

17. The pharmaceutical delivery device according to claim 16, wherein the overcap contains tamper evident features.

18. The pharmaceutical delivery device according to claim 8, wherein the container is made of polyethylene, the valveless dropper tip is made of polyethylene, and the overcap is made of polypropylene or any other suitable polymer material.

19. The pharmaceutical delivery device according to claim 9, wherein the pharmaceutical delivery device is manufactured in a translucent or transparent material.

20. The pharmaceutical delivery device according to claim 10, wherein the pharmaceutical delivery device may be used to administer medications as systemic non-invasive mucosal delivery system.

21. The pharmaceutical delivery device according to claim 10, wherein the pharmaceutical delivery device may be used to administer medications as localized mucosal delivery system.

22. The pharmaceutical delivery device according to claim 12, wherein the pharmaceutical delivery device may be used to administer medications for immediate or rate controlled delivery of medicaments.
